(19) United States
(12) Patent Application Publication
(10) Pub. No.: US 2019/0228838 A1
(43) Pub. Date: Jul. 25, 2019

(54) Tuning of Associations For Predictive Gene Scoring

(71) Applicant: McMaster University, Hamilton (CA)

(72) Inventors: Guillaume PARE, Puslinch (CA); Shihong MAO, Ancaster (CA); Wei Qxi DENG, Burlington (CA)

(73) Assignee: McMaster University, Hamilton, ON (CA)

(21) Appl. No.: 16/336,406

(22) PCT Filed: Sep. 25, 2017
(86) PCT No.: PCT/CA2017/051126

§ 371 Date: Mar. 25, 2019

Related U.S. Application Data
(60) Provisional application No. 62/399,783, filed on Sep. 26, 2016.

(51) Int. Cl. G16B 30/10; G16B 40/00; G16B 30/20; G16B 5/00

(52) U.S. Cl. CPC G16B 30/10; G16B 5/00; G16B 30/20; G16B 40/00

(57) ABSTRACT

A computer-implemented method for tuning associations between genetic variants of sequence elements of a subject genome and a target biological characteristic to a target population is described. The method notionally partitions the subject genome into discrete contiguous genome segments and derives a tuning function for each genome segment. The tuning function tunes the associations to the target population, and derivation of the tuning function for each genome segment excludes sequence elements in that same genome segment to avoid overfitting.

### TECHNICAL FIELD

The present disclosure relates to predictive gene scoring, and more particularly to tuning of associations for use in predictive gene scoring.

### BACKGROUND

Machine learning encompasses a class of methods widely used to solve complex prediction problems. Generally speaking, machine learning is a process in which computer algorithms are used to develop a predictive model based on known “training data”, which model can then be applied to new data to make predictions. Machine learning has proven particularly useful when prediction is dependent on the integration of a large number of predictor variables, including higher-order interactions, and when sizeable training datasets are available for model fitting.

One application of machine learning is to develop models for predicting biological characteristics based on variations in a particular genome. One type of variation is a single nucleotide polymorphism (SNP). Where a single nucleotide has variations at a particular position in the genome and each of those variations is present in a population beyond a negligible degree, this is considered an SNP; the variations are referred to as “alleles” for that position in the genome.

Many biological characteristics are believed to be polygenic, that is, a large number of genes influence a particular biological characteristic, but the individual effect of each gene is relatively small. Despite moderate to high narrow-sense heritability estimates for most polygenic traits, known genetic associations only explain a relatively small proportion of polygenic traits variance. It has been proposed that weak, yet undetected, associations underlie polygenic trait heritability (Yang, J., Benyamin, B., Mcevoy, B. P., Gordon, S., Henders, A. K., Dale, R., Madden, P. A., Heath, A. C., Martin, N. G., Montgomery, G. W., et al. (2010). Common SNPs explain a large proportion of the heritability for human height. Nature Genetics 42, 565-569.). Consistent with this hypothesis, polygenic scores including both strongly and weakly associated variants produce vastly superior prediction R² than the ones including only genome-wide significant variants. Thus, machine learning is well-suited to developing models for predictive gene scoring. However, an inherent risk in machine learning is “overfitting”, where the model is excessively tuned to the training data. This renders the resulting model ineffective in making predictions for data outside of the training data. Machine learning in the predictive gene scoring context is particularly susceptible to overfitting because genetic variants such as SNPs are often correlated, in many cases highly correlated, with one another. Such correlation is referred to as “linkage disequilibrium” (LD): alleles at different locations on the genome are associated with one another at a different frequency (either higher or lower) than one would expect if the associations were random. Linkage disequilibrium interferes with the ability of a machine-learning-generated predictive gene score to make generalized predictions because the model may be fitted to genetic variants which are correlated with, and hence representative of, the variables sought to be predicted and therefore overfitted.

### SUMMARY

A computer-implemented method for tuning associations between genetic variants of sequence elements of a subject genome and a target biological characteristic notionally partitions the subject genome into discrete contiguous genome segments and derives a tuning function for each genome segment to tune the associations to the target population. Derivation of the tuning function for each genome segment excludes sequence elements in that same genome segment to avoid overfitting.

In one aspect, a computer-implemented method for developing a predictive gene score for a target biological characteristic in a prediction target population comprises receiving a baseline dataset and receiving a tuning dataset. The baseline dataset comprises a first plurality of associations between genetic variants of sequence elements of a subject genome and the target biological characteristic. Similarly, the tuning dataset comprising, for a representative sample of the prediction target population, a second plurality of associations between genetic variants of the sequence elements of the subject genome and the target biological characteristic. The associations in the baseline dataset and the tuning data set are genotypic weightings representing contributions of the respective genetic variants to a value of the target biological characteristic. The method further comprises notionally partitioning the subject genome into s discrete contiguous genome segments where s≥2 so that, for each genome segment, the subject genome notionally comprises that genome segment and the remainder of the subject genome, other than and excluding that genome segment. The method obtains adjusted associations for each genome segment without overrating by, for each genome segment, using only the associations for the sequence elements in the remainder of the subject genome to derive a tuning function for that genome segment. The tuning function maps the associations in the baseline dataset for the remainder of the subject genome to respective corresponding ones of the associations in the tuning dataset for the remainder of the subject genome. The method applies the tuning function to the associations in the baseline dataset to obtain adjusted associations for the sequence elements in that genome segment, with the adjusted associations being tuned to the prediction target population. The method uses the adjusted associations to form the predictive gene score for the target biological characteristic in the prediction target population.

In some implementations, the associations for the sequence elements in the remainder of the subject genome may be used to derive a tuning function for that genome segment by deriving regression trees representing the tuning function.

In some implementations, the method may further comprise receiving at least one annotation, wherein each annotation is associated with a respective sequence element, and, for each genome segment for which the remainder of the subject genome includes a sequence element with which one of the at least one annotation is associated, using each such annotation in deriving the tuning function for that genome segment. A wide range of annotations may be used. Deriving the tuning function may comprise deriving regression trees representing the tuning function.

The present disclosure is also directed to computer-usable media embodying instructions for implementing the methods, and to computer systems programmed to implement the method.

### BRIEF DESCRIPTION OF THE DRAWINGS

These and other features will become more apparent from the following description in which reference is made to the appended drawings wherein:

FIG. 1 is a flow chart showing an exemplary method for tuning associations between genetic variants of sequence elements of a subject genome and a target biological characteristic to a target population;

FIG. 2 is a flow chart representing a first exemplary computer-implemented method for developing a predictive gene score for a target biological characteristic in a prediction target population;

FIG. 3 is a flow chart representing a second exemplary computer-implemented method for developing a predictive gene score for a target biological characteristic in a prediction target population;

FIG. 3 schematically illustrates notional partition of a subject genome into discrete contiguous genome segments;

FIGS. 4A, 4B and 4C show performance of LD-corrected gene scores simulated using 1000 Genomes Project phased haplotypes for gene score prediction R², variance and covariance, respectively;

FIGS. 5A to 5D are graphs showing comparisons among various methodologies for prediction R² for height in HRS, BMI in HRS, height in GENEVA and BMI in GENEVA, respectively;

FIG. 6 is a series of bar graphs showing relative improvement in prediction R² for various methodologies, as compared to unadjusted gene scores, for height in HRS, BMI in HRS, height in GENEVA and BMI in GENEVA; and

FIG. 7 is a block diagram of an illustrative computer system in respect of which the technology herein described may be implemented.

### DETAILED DESCRIPTION

In reviewing the present description, it is to be understood that the various methods described are particular implementations of machine learning, and hence the methods are necessarily computer-implemented methods. As such, in construing the specification and claims, it is to be understood that the use of a computer is an essential element of the claims.

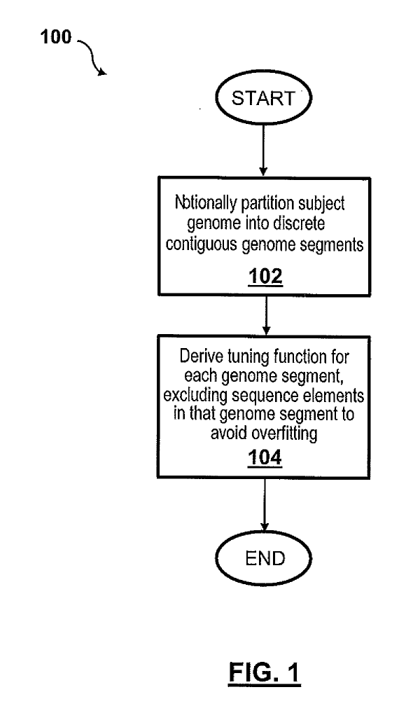

Reference is now made to FIG. 1, which is a flow chart showing, in broad outline, an exemplary method, denoted generally by reference 100, for tuning associations between genetic variants of sequence elements of a subject genome and a target biological characteristic to a target population. The subject genome may be the human genome, a non-human animal genome, or a plant genome, and the sequence elements may be, for example, single nucleotides, nucleotide sequences, or some combination thereof. The target biological characteristic may be any biological characteristic of the flora or fauna whose genome is being considered. The associations between the genetic variants of the sequence elements and the target biological characteristics are typically genotypic weightings representing contributions of the respective genetic variants to a value of the target biological characteristic.

At step 102, the method 100 notionally partitions the subject genome into discrete contiguous genome segments, and at step 104, the method 100 derives a tuning function for each genome segment. The tuning function derived at step 104 tunes the associations to the target population; the same genetic variant may have different contributions to the same biological characteristic depending on the population. For example, a particular genetic variant may have a greater contribution to height for a population of Pacific Islanders than for a population of American Indians. Importantly, derivation of the tuning function for each genome segment at step 104 excludes sequence elements in that genome segment to avoid overfitting. After step 104, the method 100 ends; the tuning functions derived at step 104 can be used to develop a predictive gene score.

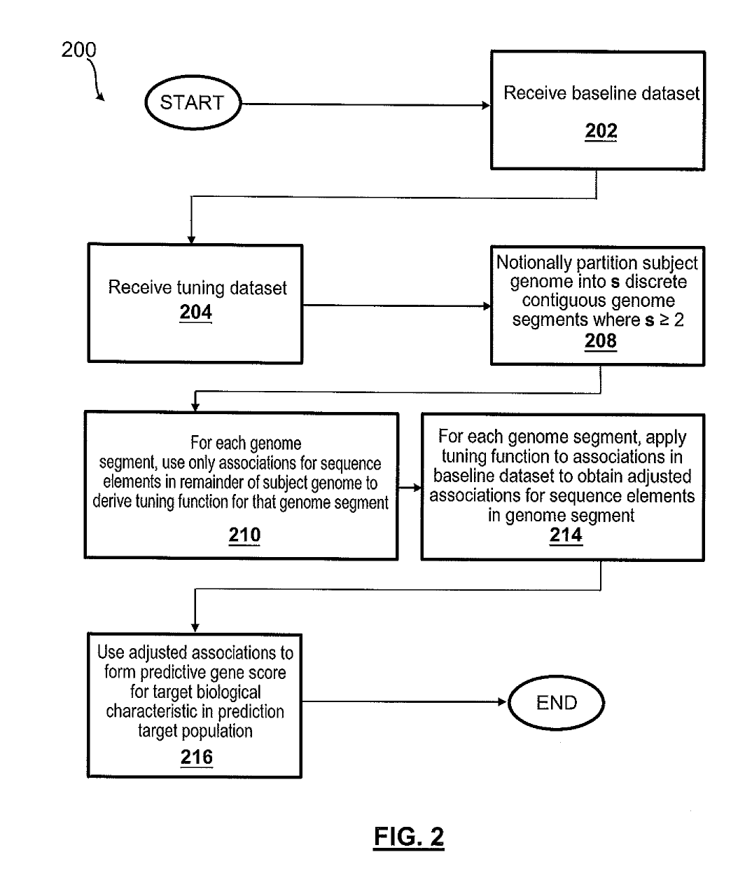

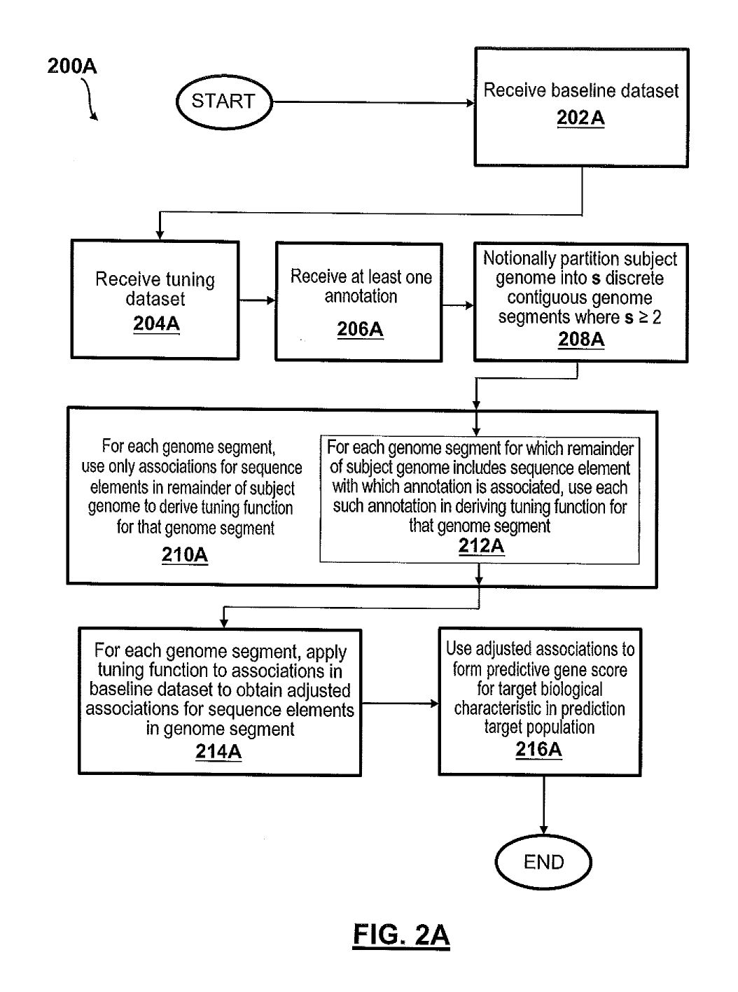

One particular exemplary implementation of the method 100 will now be described with reference to FIG. 2.

FIG. 2 shows a flow chart representing an exemplary computer-implemented method 200 for developing a predictive gene score for a target biological characteristic in a prediction target population. At step 202, the method 200 receives a baseline dataset. The baseline dataset comprises a first plurality of associations between genetic variants of sequence elements of the subject genome and the target biological characteristic. At step 204, the method 200 receives a tuning dataset. The tuning dataset comprises, for a representative sample of the prediction target population, a second plurality of associations between genetic variants of the sequence elements of the subject genome and the target biological characteristic. As noted above, the subject genome may be the human genome or that of a non-human animal or a plant, and the sequence elements may be single nucleotides, nucleotide sequences, or some combination thereof. Likewise, the target biological characteristic may be any biological characteristic of interest, and the associations between the genetic variants of the sequence elements and the target biological characteristics are genotypic weightings that represent the contributions of the respective genetic variants to the relevant value of the target biological characteristic.

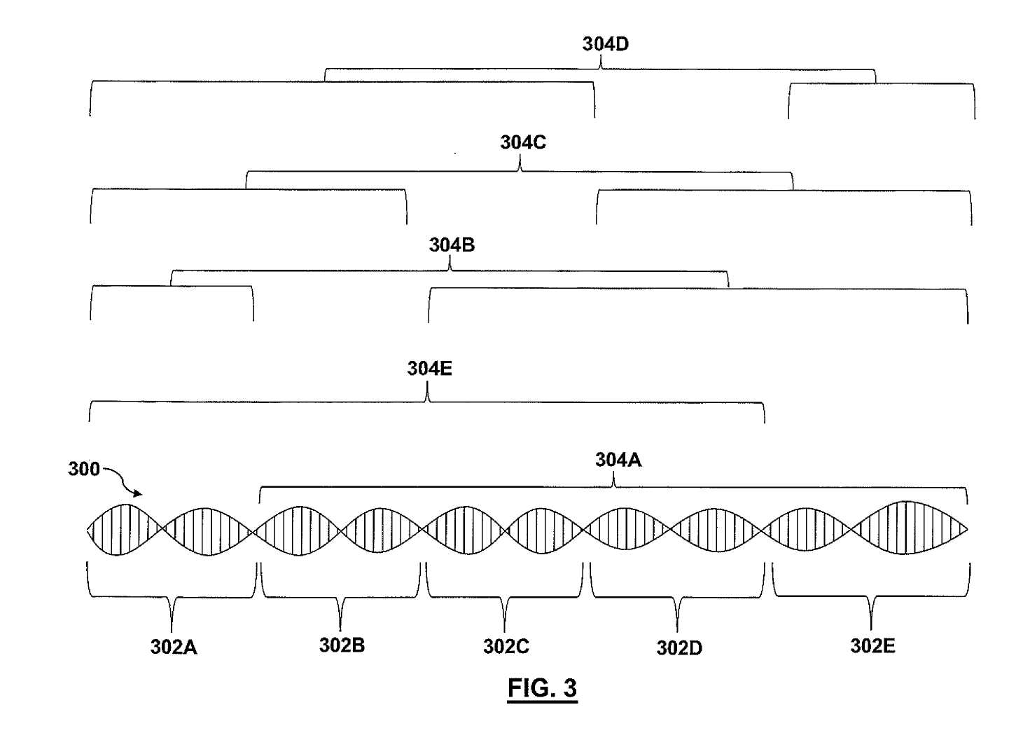

At step 208, the method 200 notionally partitions the subject genome into s discrete contiguous genome segments where s≥2 so that, for each genome segment, the subject genome notionally comprises (a) that genome segment; and (b) the remainder of the subject genome other than and excluding that genome segment. This concept is shown schematically in FIG. 3, where an exemplary subject genome 300 has been notionally divided into five genome segments 302A, 302B, 302C, 302D and 302E, that is, s=5. As can be seen in FIG. 3, for the first (leftmost) genome segment 302A, the remainder 304A of the subject genome 300 consists of the other four genome segments 302B, 302C, 302D and 302E. Similarly, for the second genome segment 302B, the remainder 304B of the subject genome 300 consists of the other four genome segments 302A, 302C, 302D and 302E, for the third genome segment 302C, the remainder 304C of the subject genome 300 consists of the other four genome segments 302A, 302B, 302D and 302E, for the fourth genome segment 302D, the remainder 304D of the subject genome 300 consists of the other four genome segments 302A, 302B, 302C and 302E and for the fifth genome segment 302E, the remainder 304E of the subject genome 300 consists of the other four genome segments 302A, 302B, 302C and 302D.

Steps 202, 204 and 208 may be carried out in any order, or substantially simultaneously. In one embodiment, step 206 (notionally partitioning the subject genome into s discrete contiguous genome segments where s≥2) is carried out by way of a predefined partition scheme defined prior to receiving the baseline dataset or the tuning dataset.

Returning now to FIG. 2, at steps 210 and 214 the method 200 obtains adjusted associations for each genome segment (e.g. the genome segments 302A, 302B, 302C, 302D and 302E shown in FIG. 3) without overfitting. This is done by, at step 210, for each genome segment, using only the associations for the sequence elements in the remainder of the subject genome to derive a tuning function for that genome segment. Thus, referring again to FIG. 3, for the first (leftmost) genome segment 302A, only the remainder 304A of the subject genome 300, that is, only the other four genome segments 302B, 302C, 302D and 302E, would be used to derive the tuning function for that genome segment 302A. Similarly, for the second genome segment 302B, only the remainder 304B of the subject genome 300 (only the other four genome segments 302A, 302C, 302D and 302E) would be used to derive the tuning function for that genome segment 302B, for the third genome segment 302C, only the remainder 304C of the subject genome 300 would be used to derive the tuning function for that genome segment 302C, and so on. Each tuning function derived at step 210 (FIG. 2), that is, the tuning function for each genome segment (e.g. the genome segments 302A, 302B, 302C, 302D and 302E shown in FIG. 3) maps the associations in the baseline dataset for the remainder 304A, 304B, 304C, 304D and 304E of the subject genome to respective corresponding ones of the associations in the tuning dataset for the remainder 304A, 304B, 304C, 304D and 304E of the subject genome. The tuning functions may be derived by, for example, deriving regression trees representing the tuning functions, as described further below.

Thus, to avoid over-fitting, the subject genome (and hence the SNPs) will be divided into distinct contiguous sets of SNPs. More particularly, since LD is relatively localized, the notional partition of the genome makes it less likely that the genetic variants in the remainder of the subject genome will correlate with the genome segment for which the tuning function is being derived, thereby limiting potential LD spillover and reducing the resulting overfitting effects. Weights of SNPs in each set are calculated using the prediction models trained on the remaining sets. For example, where s=5, if the first set comprises SNPs from chromosomes 1, 2 and part of 3, then SNPs from the remaining part of chromosome 3 and chromosomes 4 to 22 would be used to derive prediction models for SNPs in that first set. The observed regression coefficient of any single SNP in the prediction target population is thus never used directly or indirectly to derive its own gene score weight.

The notional division of the subject genome into five genome segments (n=5) is merely exemplary, and the subject genome may be notionally divided into any number of genome segments, i.e. s≥2. If the value of s is set too high, the sequence elements in adjacent genome segments will be very close to one another, which may undermine the desired reduction of LD spillover since there may be LD between the sequence elements in adjacent or nearby genome segments. Therefore, the value of s is preferably less than 2000, more preferably less than 1000, still more preferably less than 100, still even more preferably less than 50 and yet still even more preferably less than 20.

At step 214, for each genome segment (e.g. the genome segments 302A, 302B, 302C, 302D and 302E shown in FIG. 3) the method 200 applies the tuning function to the associations in the baseline dataset to obtain adjusted associations for the sequence elements in that genome segment. These adjusted associations are genotypic weightings that represent the contributions of the respective genetic variants to the relevant value of the target biological characteristic, and which genotypic weightings are tuned to the prediction target population.

At step 216, the method 200 uses the adjusted associations to form the predictive gene score for the target biological characteristic in the prediction target population, after which the method 200 ends. While step 210 above will limit the effect of linkage disequilibrium in deriving the tuning functions, where there are multiple genetic variants in a predictive gene score, it is advantageous to take linkage disequilibrium into account when using the adjusted associations to form the predictive gene score. There is a distinction between avoiding the overfitting effects of linkage disequilibrium to derive the tuning functions, and correcting the genotypic weightings (resulting from the tuning functions) to account for linkage disequilibrium. Therefore, in preferred embodiments, step 216 includes adjustment based on linkage disequilibrium, as described further below. After step 216, the method 200 ends.

The most popular linkage disequilibrium adjustment heuristic is based on linkage disequilibrium (LD) pruning of SNPs. The LD pruning heuristic prioritizes the most significant associations up to an empirically determined p-value threshold and prunes the remaining SNPs based on LD (International Schizophrenia, C., Purcell, S. M., Wray, N. R., Stone, J. L., Visscher, P. M., O'Donovan, M. C., Sullivan, P. F., and Sklar, P. (2009). Common polygenic variation contributes to risk of schizophrenia and bipolar disorder. Nature 460, 748-752). This “pruning and thresholding” (P+T) approach has the advantage of being simple and computationally efficient, but discards some information because of LD pruning. To remediate this issue, a novel method was recently proposed that uses LD information from an external reference panel to infer the mean causal effect size using a Bayesian approach (LDpred) (Vilhjalmsson, B. J., Yang, J., Finucane, Gusev, A., Lindstrom, S., Ripke, S., Genovese, G., Loh, P. R., Bhatia, G., Do, R., et al. (2015). Modeling Linkage Disequilibrium Increases Accuracy of Polygenic Risk Scores. American Journal of Human Genetics 97, 576-592). While the latter method has been shown to improve prediction R², a further gain in prediction R² may be made by tuning associations through the integration of SNP annotations using machine-learning algorithms.

One particular implementation of a method according to the present disclosure leverages the large number of SNP annotations available to improve the weights of SNPs contributing to the gene score. Depending on the nature of the SNP annotations used as predictor variables, accuracy may be improved where the annotations are independent of the tuning dataset; in other cases the annotations need not be independent of the tuning dataset (e.g. allele frequency).

Reference is now made to FIG. 2A, which shows a flow chart representing a further exemplary computer-implemented method 200A for developing a predictive gene score for a target biological characteristic in a prediction target population. The method 200A shown in FIG. 2A represents a particular implementation of the method 200 shown in FIG. 2, and hence the same reference numerals, with the additional suffix “A”, are used to denote corresponding steps. The method 200A shown in FIG. 2A differs from the method 200 shown in FIG. 2 in that the method 200A makes use of SNP annotations. Thus, the method 200A shown in FIG. 2A includes an additional step 206A of receiving at least one annotation, with each annotation being associated with a respective sequence element in the subject genome. The annotation(s) may be, for example, one or more of the following:
(1) Association of genetic variants with other traits, including but exclusive to association with health outcomes (e.g. coronary artery disease, asthma, cancer, etc.), individual characteristics (e.g. height, weight, blood pressure, hair colour, etc.), gene level of expression (eQTL) in relevant tissue(s), DNA methylation or other biomarkers;
(2) Allele frequency of the variant in relevant populations;
(3) Assessment of the functional role of each variant or of other variants in linkage disequilibrium (i.e. correlated) with it, including but not restricted to:
   Coding variant (i.e. inducing a change in the amino acid sequence of a protein) and corresponding estimate of the disruptiveness of the change in amino acid sequence;
   Variant disrupting or creating a transcription factor binding site;
   Variant disrupting or creating a messenger RNA splice site;
   Variant disrupting or creating a site for epigenetic regulation such as but not exclusive to DNA methylation;
   Variant changing the sequence of a non-coding RNA;
   Variant changing the binding site of a microRNA;
   Variant located in a DNase I hypersensitive region in relevant tissue(s);
(4) Measure of evolutionary conservation or evolutionary selection of the variant or other variants in linkage disequilibrium with it;
(5) Measure of linkage disequilibrium of the variant with other neighboring variants;
(6) Assessment of the functional role of the genetic region in which the variant is located irrespective of linkage disequilibrium with the variant, such as regional association with other traits;
(7) Summary measure of the functional potential of a variant, which may include a score derived from one or more annotations.

Steps 202A, 204A, 206A and 208A may be performed in any order, or substantially simultaneously.

In the method 200A shown in FIG. 2A, step 210A includes a sub-step 212A of, for each genome segment for which the remainder of the subject genome includes a sequence element with which one of the annotations is associated, using each such annotation in deriving the tuning function for that genome segment. Step 212A is shown as a distinct sub-step only for purposes of schematic illustration, and typically the use of the annotations to derive the tuning functions will be fully integrated into the overall calculations used to derive the tuning functions at step 210A.

One exemplary implementation of the method 200A uses the univariate regression coefficients from external meta-analysis summary association statistics (i.e. the baseline dataset) as a starting point, and uses the tuning dataset to update these external univariate regression coefficients with respect to a target population by way of boosted regression trees, integrating a variety of SNP annotations.

Boosted regression trees are powerful and versatile methods for continuous outcome prediction (Hastie, T., Tibshirani, R., and Friedman, J. H. (2009). The elements of statistical learning: data mining, inference, and prediction. (New York, N.Y.: Springer).) and thus well-suited to updating the SNP weights in a gene score. The exemplary method described herein uses boosted regression trees to adjust summary association statistics regression coefficients in order to improve the prediction R² in a prediction target population. Regression coefficients from large meta-analyses are implicitly assumed to provide the best initial estimates and regression trees “tune” them based on the regression coefficients observed in the prediction target population as well as relevant SNP annotations; thus, the regression trees serve as tuning functions.

As noted above, in preferred embodiments the adjusted associations (i.e. the tuned genotypic weightings) are corrected for LD to produce the final predictive gene score.

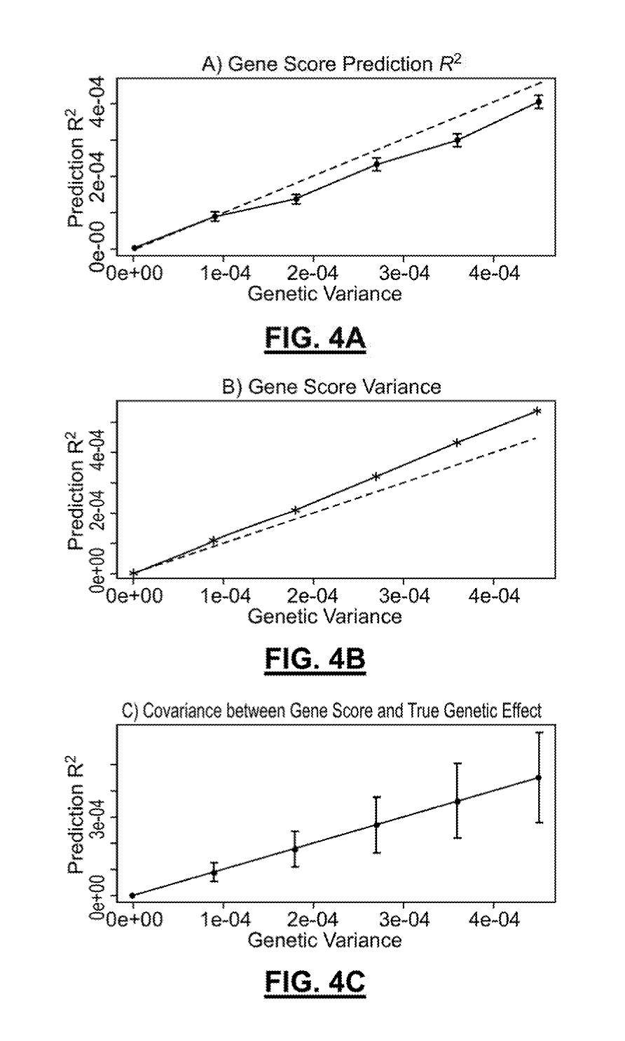

It is advantageous to correct the adjusted associations (gene score weights) for LD when including multiple SNPs in a gene score, unless SNPs are first LD pruned. One exemplary correction method is based on the sum of pairwise LD r² of each SNP over neighboring SNPs. The gene score weight of each SNP is divided by the corresponding sum of r². To illustrate with a simple example, if five SNPs are in perfect LD (r²=1) with each other but in linkage equilibrium with all other SNPs (r²=0), then the gene score weights of these five SNPs would be divided by five. As all five SNPs are included in the gene score and the effect of all five SNPs summed, the corrected weight contributions are equivalent to including a single SNP without correction. This also explains why it is necessary to apply the LD correction only after adjusting SNP weights with boosted regression trees as otherwise important information on strength of association of individual SNPs would be lost. LD is summed over SNPs included in the gene score, such that the correction is specific to the set of SNPs included in a given gene score. When the genetic effects are strictly additive (i.e. no haplotype or interaction effect), the resulting gene score provides an unbiased estimate of the underlying genetic variance although at a tradeoff of increased gene score variance as compared to the “true” unobserved genetic model (see FIG. 4). The loss in prediction R² resulting from increased gene score variance was estimated at ˜12% in simulations using phased haplotypes from the 1000 Genomes (1000G) Project, as described further below.

An exemplary method to correct summary association regression coefficients for LD in such a way that all SNPs can be included in a gene score, irrespective of LD, will now be described. Genotypes for n individuals at in SNPs are given by a matrix

X_{n×m}=[x₁ X₂ . . . xₙ]^{T}

with each column vector x₁, x₂, . . . , xₙ representing the coded genotypes for an individual. Without loss of generality, it is assumed that each column of X (i.e. genotypes for a single SNP) to be standardized to have mean 0 and variance 1. For a standardized quantitative trait y with mean 0 and variance 1, the underlying linear model can be expressed as:

y=Xβ+ε (eq. 1)

β is a vector of true genetic effects that are fixed across individuals but random across SNPs, with mean 0 and covariance matrix σ²I such that the total expected genetic variance

Rₜᵣᵤₑ²=E(β^{T}X^{T}Xβ)=tr(X^{T}X)σ²=mσ²

and ε the error term with mean 0 and covariance (1−mσ²)I so that the covariance of y is I. Let r²_{d,k} denote the pairwise linkage disequilibrium (r²) between the d^{th} and k^{th} SNPs. The LD adjustment (η_{d}) for the d^{th} SNP is defined by the sum of r² between the d^{th} SNP and the 100 SNPs upstream and downstream:

$${η}_{d} = {\sum }_{k = d - 100}^{k = d + 100} {r}_{d , k}^{2} \qquad ( eq . 2 )$$

with a distance of 100 SNPs assumed sufficient to ensure linkage equilibrium (other values might be used). Including only SNPs that are part of the gene score in the calculation of η_{d}, the LD-corrected regression coefficients are given by:

$${\tilde{b}}_{d} = \frac{{b}_{d}^{*}}{{η}_{d}} \qquad ( eq . 3 )$$

where b_{d}* is the regression coefficient commonly reported in genome-wide association studies (GWAS) meta-analysis (assumed to have been standardized for allele frequency). Given xᵢ, the genotypes of n SNPs for the i^{th} individual, the gene score g(xᵢ) is:

$$g ( {x}_{i} ) = {x}_{i}^{T} \tilde{b} = {\sum }_{d} \times {x}_{i , d} \frac{{b}_{d}^{*}}{{η}_{d}} = {x}_{i}^{T} {Cb}^{*} \qquad ( eq . 4 )$$

where C is an m×m diagonal matrix with entries

$$( \frac{1}{{η}_{1}} , \frac{1}{{η}_{2}} , … , \frac{1}{{η}_{m}} ) .$$

The prediction R² of the gene score in the target population is expressed as:

$${R}^{2} = \frac{{Cov ( g ( X ) , y )}^{2}}{Var ( g ( X ) ) Var ( y )} \qquad ( eq . 5 )$$

The expected value can be approximated by:

$$E [ {R}^{2} ] = E [ \frac{{Cov ( g ( X ) , y )}^{2}}{Var ( g ( X ) ) Var ( y )} ] = E [ \frac{{Cov ( g ( X ) , y )}^{2}}{Var ( g ( X ) )} ] \sim \frac{E [ {Cov ( g ( X ) , y )}^{2} ]}{E [ Var ( g ( X ) ) ]} \sim \frac{{E [ Cov ( g ( X ) , y ) ]}^{2}}{E [ Var ( g ( X ) ) ]} \qquad ( eq . 6 )$$

and leading to

$$E [ {R}^{2} ] \sim \frac{{E [ Cov ( g ( X ) , y ) ]}^{2}}{E [ Var ( g ( X ) ) ]} = \frac{{( {R}_{true}^{2} )}^{2}}{E [ Var ( g ( X ) ) ]} < {R}_{true}^{2} \qquad ( eq . 7 )$$

by deriving the following relations:
(1) E[Cov(g(X),y)]=Rₜᵣᵤₑ², implying the covariance between the gene score and the trait is an unbiased estimator of the true genetic variance; and
(2) E[Var(g(X))]>Rₜᵣᵤₑ² and thus E[R²]<Rₜᵣᵤₑ², implying the expected prediction R² must be bounded above by the true genetic variance.

With respect to the first relation, that is, E[Cov(g(X),y)]=Rₜᵣᵤₑ², which implies that the covariance between the gene score and the trait is an unbiased estimator of the true genetic variance, the sample covariance of the gene score with the observed y in the target sample is given by:

$$Cov ( g ( X ) , y ) = \frac{1}{n} {\sum }_{i = 1}^{n} g ( {x}_{i} ) {y}_{i} = \frac{1}{n} {( {XCb}^{*} )}^{T} ( X β + e ) = \frac{1}{nN} {( XC ( {X}^{* T} {y}^{*} ) )}^{T} ( X β + e ) = \frac{1}{nN} {( XC ( {X}^{* T} {y}^{*} ) )}^{T} ( X β + e ) = \frac{1}{nN} ( {β}^{T} {X}^{* T} {X}^{*} {CX}^{T} X β + {e}^{* T} {X}^{* T} {X}^{*} {CX}^{T} e + {β}^{T} {X}^{* T} {X}^{*} {CX}^{T} e + {e}^{* T} {X}^{* T} {X}^{*} {CX}^{T} X β ) \qquad ( eq . 8 )$$

where e* and e are the residual error in the unobserved population used to derive summary association statistics (i.e. the baseline dataset) and the prediction target population, respectively. The reported b* in genome-wide association studies (GWAS) meta-analysis are constructed to estimate the univariate regression coefficients from the otherwise unobserved genotype matrix X*_{N×m} and quantitative trait y*:

$${b}^{*} \sim \frac{{X}^{* T} y}{N} = \frac{{X}^{* T} ( {X}^{*} β + {e}^{*} )}{N} = \frac{{X}^{* T} {X}^{*}}{N} β + \frac{{X}^{* T} {e}^{*}}{N} \qquad ( eq . 9 )$$

Assume the prediction target population is independent of the meta-analysis, i.e. e* and e are independent, we have the expected value of the quadratic forms in (eq. 8):

$$E [ Cov ( g ( X ) , y ) ] = \frac{1}{nN} E [ ( {β}^{T} {X}^{* T} {X}^{*} {CX}^{T} X β + {e}^{* T} {X}^{* T} {X}^{*} {CX}^{T} e + {β}^{T} {X}^{* T} {X}^{*} {CX}^{T} e + {e}^{* T} {X}^{* T} {X}^{*} {CX}^{T} X β ) ] = \frac{1}{nN} E [ {β}^{T} {X}^{* T} {X}^{*} {CX}^{T} X β ] = tr ( {β}^{T} \frac{{X}^{* T} {X}^{*}}{N} C \frac{ {X}^{T} X}{n} β ) = {σ}^{2} tr ( \frac{{X}^{* T} {X}^{*}}{N} C \frac{{X}^{T} X}{n} ) = {σ}^{2} m = {R}_{true}^{2} \qquad ( eq . 10 )$$

This equality holds for all positive definite matrices of the form

$$\frac{{X}^{* T} {X}^{*}}{N} C \frac{ {X}^{T} X}{n} ,$$

assuming the LD structure in the two populations is identical. It has thus been shown that Cov(g(X),y) is an unbiased estimator of the true genetic variance.

For the second relation, E[Var(g(X))]>Rₜᵣᵤₑ² and thus E[R²]<Rₜᵣᵤₑ², implying the expected prediction R² must be bounded above by the true genetic variance, the denominator in (eq. 7), E[Var(g(X))], can be shown to be greater than Rₜᵣᵤₑ²:

Rₜᵣᵤₑ²=E[Cov(g(X),y)]=E[Cov(g(X), Xβ)]

E[Cov(g(X), Xβ)]≤E[{square root over (Var(g(X))Var(Xβ))}]=E[{square root over (Var(g(X))Rₜᵣᵤₑ²)}]

and thus:

Rₜᵣᵤₑ²≤E[{square root over (Var(g(X))Rₜᵣᵤₑ²)}] and \sqrt{Rₜᵣᵤₑ²}≤E[{square root over (Var(g(X)))}]

giving:

E[Var(g(X))]≥E[{square root over (Var(g(X)))}]²≥Rₜᵣᵤₑ² (eq. 11)

From the above inequality, it can be concluded that E[Var(g(X))] will always be greater or equal than the true genetic variance.

The above-described LD correction method offers a tradeoff between bias and variance, whereby genetic variance estimates are unbiased as E[Cov(g(X),y)]=Rₜᵣᵤₑ² but E[Var(g(X))]≥Rₜᵣᵤₑ² implying that R²≤Rₜᵣᵤₑ². It can be shown that R²=Rₜᵣᵤₑ² in simple cases where pairwise r² LD is either 0 or 1 and summary association statistics are derived from an asymptotically large sample. However, in more common scenarios with partial LD R²<Rₜᵣᵤₑ² reflecting the loss of information when, for example, two SNPs are in partial LD and have true genetic effects with opposite directions. To assess the importance of this effect in plausible situations, certain simulations were carried out. In particular, 5,000 individuals were simulated for 450 contiguous SNPs using phased haplotypes from the 1000 Genomes Project. The genetic effect of each SNP was randomly selected from a normal distribution according to a pre-defined, unobserved, true regional genetic variance that assumed genome-wide heritability varying from 0 to 0.5. For each genetic variance set-point, 1,000 simulations were completed and a gene score incorporating LD correction derived. The average (±SD) gene score prediction R², gene score variance and covariance between gene score and true (unobserved) genetic effect as a function of time genetic variance were calculated, as shown in FIGS. 4A (prediction R²), 4B (variance) and 4C (covariance). In particular, 5,000 individuals were simulated for 450 contiguous SNPs using phased haplotypes from the 1000 Genomes Project. The genetic effect of each SNP was randomly selected from a normal distribution according to a pre-defined, unobserved, true regional genetic variance that assumed genome-wide heritability varying from 0 to 0.5. For each genetic variance set-point, 1,000 simulations were completed and a gene score incorporating LD correction derived. Each point in FIGS. 4A, 4B and 4C represents the average (±SD) of these 1,000 simulations. These simulations support the conclusion that (1) LD-corrected gene scores are unbiased estimators of true genetic variance (i.e. E[Cov(g(X),y)]=Rₜᵣᵤₑ²), and (2) variance of gene score is indeed higher than true genetic variance. Based on these simulations, the loss of information was estimated at ˜12%, or in other words gene score prediction R² was on average ˜88% of true genetic effect.

Some exemplary applications of the method 200A will now be described.

In one instance, an implementation of the method 200A using boosted regression trees was applied to the prediction of height in the Health Retirement Study (HRS; N=7,776) using Genetic Investigation of Anthropometric Traits (GIANT) consortium summary association statistics (Vilhjalmsson et al., supra; Speliotes, E. K., Willer, C. J., Berndt, S. I., Monda, K. L., Thorleifsson, G., Jackson, A. U., Lange Allen, H., Lindgren, C. M., Luan, J., Magi, R., et al. (2010). Association analyses of 249,796 individuals reveal 18 new loci associated with body mass index. Nature Genetics 42, 937-948.) on 681K SNPs (FIG. 1). Thus, the GIANT summary association statistics served as the baseline dataset received at step 202, and the HRS population was the prediction target population, with the HRS dataset serving as the tuning dataset received at step 204.

Publically available genome-wide data that are part of the Health Retirement Study (HRS; dbGaP Study Accession: phs000428.v1.p1) generated using the Human Omni2.5-Quad BeadChip (Illumina) were used. HRS quality control criteria were used for filtering of both genotype and phenotype data, namely:
(1) SNPs and individuals with missingness higher than 2% were excluded;
(2) related individuals were excluded;
(3) only participants with self-reported European ancestry and genetically confirmed by principal component analysis were included;
(4) individuals for whom the reported sex does not match their genetic sex were excluded;
(5) SNPs with Hardy-Weinberg equilibrium p<1×10⁻⁶ were excluded; and
(6) SNPs with minor allele frequency lower than 0.02 were removed.

The final dataset included 7,776 European participants genotyped for 681,516 SNPs. Height and BMI was adjusted for age and sex in all analyses; and to further mitigate the effect of outliers, values outside the 1^{st} and 99^{th} percentile range were removed. All analyses were adjusted for the first 10 genetic principal components unless stated otherwise. HRS was not part of the GIANT meta-analysis of height and BMI (Berndt, S. I., Gustafsson, S., Magi, R., Ganna, A., Wheeler, E., Feitosa, M. F., Justice, A. E., Monda, K. L., Croteau-Chonka, D. C., Day, F. R., et al. (2013), Genome-wide meta-analysis identifies 11 new loci for anthropometric traits and provides insights into genetic architecture. Nat Genet 45, 501-512. Lango Allen, H., Estrada, K., Lettre, G., Berndt, S. I., Weedon, M. N., Rivadeneira, F., Willer, C. J., Jackson, A. U., Vedantam, S., Raychaudhuri, S., et al. (2010). Hundreds of variants clustered in genomic loci and biological pathways affect human height. Nature 467, 832-838.).

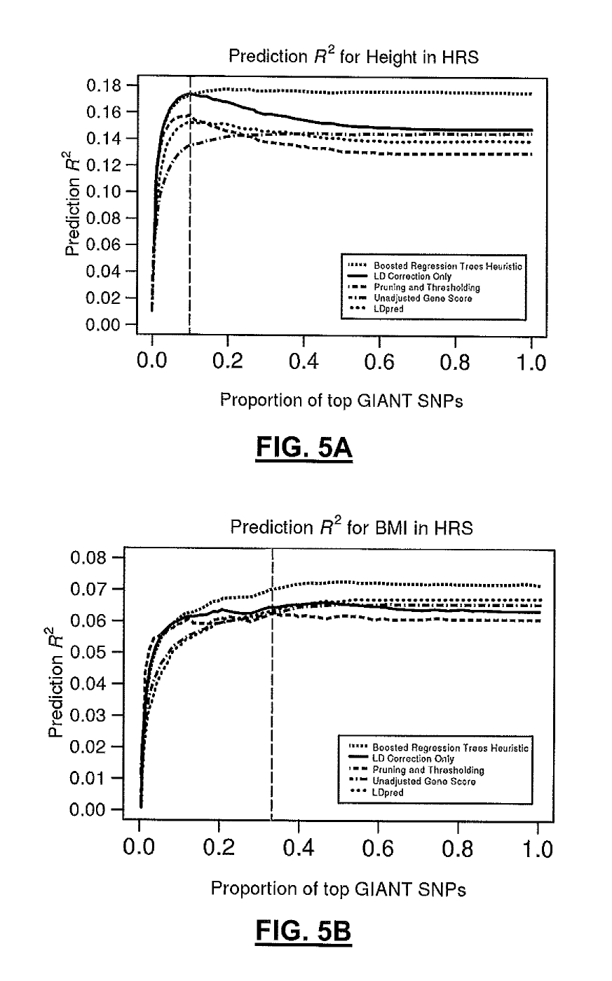

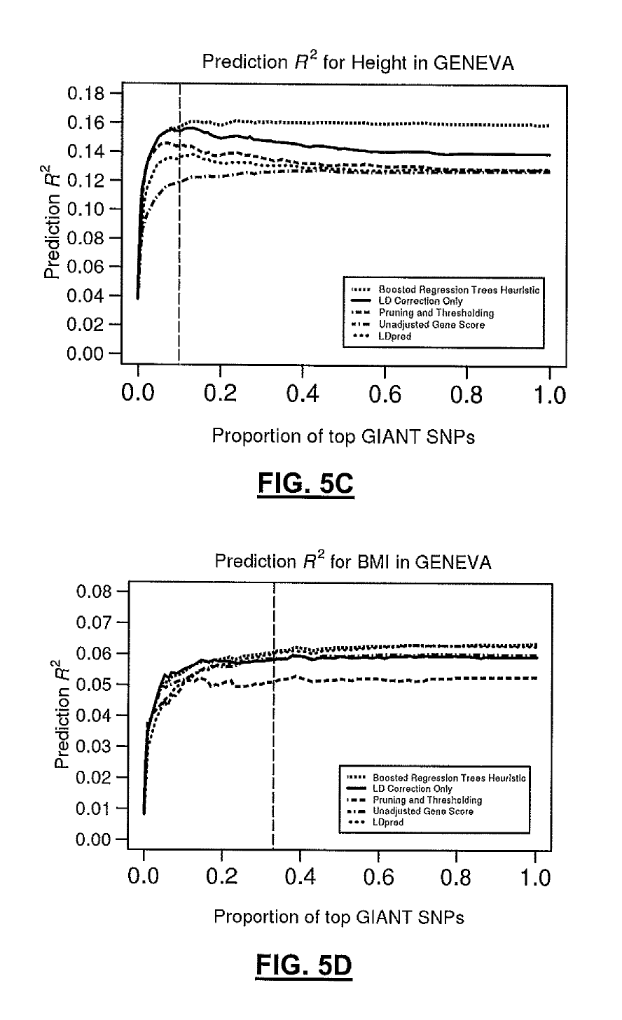

Since HRS is not part of the GIANT consortium, the reference and target populations (i.e. baseline dataset and tuning dataset, respectively) can be assumed to be independent. As recently proposed (Chen, C. Y., Han, J., Hunter, D. J., Kraft, P., and Price, A. L. (2015). Explicit Modeling of Ancestry Improves Polygenic Risk Scores and BLUP Prediction. Genet Epidemiol 39, 427-438.), principal components were added to the model and included in the prediction R². A total of 59 prediction variables were considered for inclusion in the boosted regression trees models and after initial pruning 32 remained. Prediction R² of gene score derived using the method 200A was 0.176, corresponding to 46.6% of total polygenic genetic variance estimated in HRS using variance component models (Yang, J., Lee, S. H., Goddard, M. E., and Visscher, P. M. (2011). GCTA: a tool for genome-wide complex trait analysis. American Journal of Human Genetics 88, 76-82.) (i.e. 0.377). As shown in FIGS. 5A to 5D, this compared advantageously to the optimal prediction R² obtained with P+T (0.158; 138K SNPs), LDpred (0.139) or an unadjusted gene score (0.145). The performance of the above-described LD correction method alone, without boosted regression trees, was also tested. Application of the LD correction method on its own was associated with a maximal prediction R² of 0.148, which rivaled other methods but remained inferior to the implementation of the method 200A using regression trees.

Table 1 below shows the relative influence of predictor variables for height and BMI. The top five predictor variables for the gene score weights of height and BMI in HRS are listed, along with their relative influences on the regression trees models in terms of square error loss. Results are given for the first SNP set, corresponding to all SNPs from the start of the first chromosome to SNP rs1032726 on chromosome 3, and are representative of the four other SNP sets.

TABLE 1
Relative Influence of Predictor Variables for Height and BMI

| | |
|---|---|
| PREDICTOR VARIABLES | Relative Influence (%) |

HEIGHT

| | |
|---|---|
| BMI Regional Association | 10.1 |
| Height p-value (GIANT) | 9.2 |
| Triglycerides Regional Association | 8.1 |
| LD Score | 7.3 |
| LDL Cholesterol Regional Association | 7.2 |

BMI

| | |
|---|---|
| BMI Regression Coefficients (GIANT) | 48.8 |
| LDL Cholesterol Regional Association | 6.7 |
| Total Cholesterol Regional Association | 5.5 |
| LD Score | 5.1 |
| Systolic Blood Pressure Regional Association | 4.6 |

An advantage of boosted regression trees models is the potential to uncover unexpected relationships between a trait of interest and predictor variables. Indeed, it is possible to evaluate the relative influence of predictor variables on squared error reduction, even in the presence of higher-order interactions. For example, the most influential variable for the prediction of height was BMI regional genetic variance, a measure of regional genetic association that has been recently described (Pare, G., Mao, S., and Deng, W. Q. (2016). A method to estimate the contribution of regional genetic associations to complex traits from summary association statistics. Sci Rep 6, 27644.). Stronger BMI genetic associations in a region spanning 100 SNPs upstream and downstream of a given SNP predicted stronger height gene score weights, which can be interpreted as a higher likelihood of true association with height for SNPs whose neighbors are also associated with BMI. The second most influential variable was the association p-value of each SNP itself with height in GIANT (see Table 1 above), whereby stronger GIANT associations were more likely to be preserved in HRS. LDscore (Bulik-Sullivan, B. K., Loh, P. R., Finucane, H. K., Ripke, S., Yang, J., Schizophrenia Working Group of the Psychiatric Genomics, C., Patterson, N., Daly, M. J., Price, A. L., and Neale, B. M. (2015). LD Score regression distinguishes confounding from polygenicity in genome-wide association studies. Nature Genetics 47, 291-295.), a measure of LD with neighboring SNPs in 1000G data (i.e. not restricted to SNPs included in the gene score), was the fourth most predictive variable, as would be expected in polygenic models of inheritance.

The performance of an implementation of the method 200A using boosted regression trees was also tested for prediction of body mass index (BMI) in the HRS. The boosted regression trees models included 31 predictor variables which were used to update gene score weights. The resulting gene score had a prediction R² of 0.071, outperforming the prediction R² of unadjusted gene score (0.065), P+T (0.062) and LDpred (0.067) (FIG. 5B). The boosted regression tree implementation of the method 200A accounted for 44.4% of the total polygenic variance, which was estimated at 0.160 for BMI in HRS. The most influential variable was the association of each SNP itself with BMI in GIANT (see Table 1 above). LDL cholesterol, total cholesterol and systolic blood pressure regional genetic variance were the second, third and fifth most influential predictor variables, respectively, while LDscore came fourth. The use of LD correction alone outperformed P+T but was inferior to other methods.

Consistent with the notional partitioning of the subject genome at step 208A, for any given SNP, the regression coefficient observed in HRS was not used to determine its own gene score weight. Nonetheless, regression coefficients of other SNPs in HRS were used, raising the issue of transferability to other populations. Therefore, gene scores derived from HRS in Gene-Environment Association Studies (GENEVA) participants of European descents (N=2,537) were tested. GENEVA is a multi-site collaborative program initiated in 2006 as a part of the National Institutes of Health (NIH) Genes, Environment and Health Initiative (GEI) that aims to accelerate the understanding of genetic and environmental contributions to health and disease (Cornelis, M. C., Agrawal, A., Cole, J. W., Hansel, N. N., Barnes, K. C., Beaty, T. H., Bennett, S. N., Bierut, L. J., Boerwinkle, E., Doheny, K. F., et al. (2010). The Gene, Environment Association Studies consortium (GENEVA): maximizing the knowledge obtained from GWAS by collaboration across studies of multiple conditions. Genet Epidemiol 34, 364-372.). The GENEVA Diabetes study is part of the GENEVA study and aims to better understand the etiology of diabetes through investigation of gene-environment interactions in participants of the Nurses' Health Study (NHS) and Health Professionals Follow-up Study (HPFS). Imputed SNP genotypes from 2,557 participants of the GENEVA Diabetes study are available (Qi, Q., Liang, L., Doria, A., Hu, F. B., and Qi, L. (2012). Genetic predisposition to dyslipidemia and type 2 diabetes risk in two prospective cohorts. Diabetes 61, 745-752.) and were downloaded from dbGap (phs000091.v2.p1). Briefly, genotyping was performed using the Affymetrix Human SNP Array 6.0 and Birdseed calling algorithm, with standard quality control including sex chromosome abnormalities, sample identity, Hardy-Weinberg equilibrium (p-value>1×10⁻⁴), and call rate (>98%). MACH (http://www.sph.umich.edu/csg/abecasis/mach) was used to impute SNPs on chromosomes 1-22, with National Center for Biotechnology Information build 36 of phase II HapMap CEU data (release 22) as the reference panel. Height and BMI were adjusted for age and sex in all analyses. Similarly to HRS, values outside the 1^{st} and 99^{th} percentile range were removed for each of height and BMI. GENEVA Diabetes is not part of the GIANT meta-analysis of height and BMI (see Berndt et al. and Lango Allen et al., supra.) (a different subset of the NHS was included in GIANT e.g. (Hunter, D. J., Kraft, P., Jacobs, K. B., Cox, D. G., Yeager, M., Hankinson, S. E., Wacholder, S., Wang, Z., Welch, R., Hutchinson, A., et al. (2007). A genome-wide association study identifies alleles in FGFR2 associated with risk of sporadic postmenopausal breast cancer. Nature Genetics 39, 870-874.) and HPSF was not included at all).

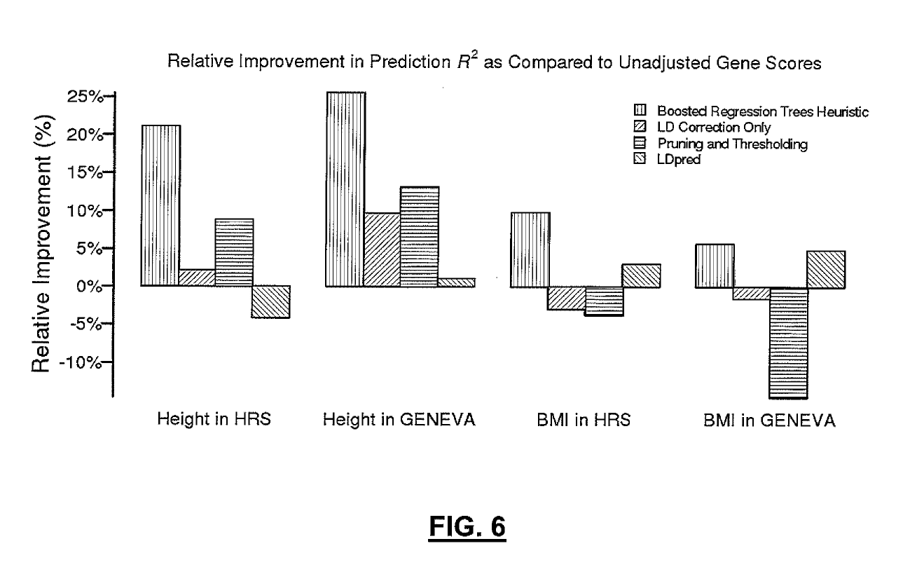

For each method, the optimal gene score derived in HRS was tested in GENEVA without any further fitting or adjustment. Consistent with HRS results, the boosted regression tree implementation of the method 200A outperformed other methods for both height and BMI, albeit the difference was less pronounced for BMI, as shown in FIGS. 5A to 5D and FIG. 6. The relative increase in prediction R² over unadjusted gene score was 25.5% for height and 5.6% for BMI. The gene score prediction R² of 0.160 and 0.063 explained 31.0% and 43.8% of the total estimated polygenic variance of height and BMI in GENEVA, respectively.

Pruning and thresholding (P+T) polygenic scores were derived using the “clump” function of PLINK (Purcell, S., Neale, B., Todd-Brown, K., Thomas, L., Ferreira, M. A., Bender, D., Mailer, J., Sklar, P., de Bakker, P. I., Daly, M. J., et al. (2007). PLINK: a tool set for whole-genome association and population-based linkage analyses. American Journal of Human Genetics 81, 559-575.) with an LD r² threshold of 0.2 and testing p-value thresholds in a continuous manner from the most to the least significant association. LDpred adjusts genome-wide association studies (GWAS) summary statistics for the effects of linkage disequilibrium, providing re-weighted effect estimates that are then used in gene scores (Vilhjalmsson et al., supra.). LDpred was run as recommended by the authors, including both the data synchronization and LDpred steps. LDpred requires specification of the fraction of SNPs assumed to be causal. For each model, causal fractions of 1 (infinitesimal), 0.3, 0.1, 0.03, 0.01, 0.003, 0.001, 0.0003, 0.0001 were tested as recommended. Results are presented using the causal fraction giving the best results only. A heritability estimate is also required by the algorithm and is estimated from summary association statistics by LDpred. As a sensitivity analysis, heritability estimates given by the variance component models in HRS were additionally used. Results were consistent and only the default option is shown. Polygenic genetic variance (i.e. narrow sense heritability) was estimated for height and BMI in HRS and GENEVA using variance components, as implemented in GCTA (Yang, J., Lee, S. H. et al., supra). All LD measures or related estimates used described herein were derived from HRS genotypes.

The exemplary boosted regression trees implementation of the method 200A led to significant improvements in prediction R² as compared to existing methods. The methods 200 and 200A leverage the large number of genetic variants reported in genome-wide association studies (GWAS) to train boosted regression trees models. Regression trees can capture nonlinear effects and higher-order interactions while the boosting algorithm combines individually weak predictors to produce a strong classifier that enables a better prediction of genetic effects.

Boosted regression trees are powerful and versatile methods that combine otherwise weak classifiers to produce a strong learner for continuous outcome prediction (Hastie et al., supra). They are thus well-suited for prediction of SNP gene score weights (ŵ_{pred}), where each fitted ŵ_{pred} gives the contribution of individual SNPs to the final gene score. The dependent variable used in boosted regression trees is constructed following:

w_{pred}*=(β_{obs}−βₑₓₜ)sign(βₑₓₜ)

In other words, w_{pred}* is derived to reflect the amount of deviation towards the null hypothesis of no association in the target population (β_{obs}) with respect to the externally derived summary association statistics estimates (βₑₓₜ). When w_{pred}=0 then β_{obs}=βₑₓₜ, implicitly assuming regression coefficients from large meta-analyses provide the best initial estimates. While some information is lost because of this construct, the resulting estimates are more robust and the overall performance improved. Boosted regression trees can be expressed as

E(w_{pred}*|X₁, X₂, . . . Xₖ)˜α+ƒ(X)

where ƒ is a regression function of trees with input variables X=(X₁, X₂, . . . Xₖ). The gradient boost algorithm aims to minimize the expected square error loss with respect to ƒ iteratively on weighted versions of the training data. In the exemplary method 200A shown in FIG. 2A, SNP annotations are included as inputs (i.e. X₁, X₂, . . . Xₖ) and their contributions are concordantly transformed to reflect the strength of association, irrespective of the direction of effect (e.g. by taking the absolute value). Importantly, consistent with step 208A of the method 200A, SNPs are divided into 5 distinct sets of contiguous SNPs (to avoid LD spillover) and fitted ŵ_{pred}* which are used in calculation of the actual gene scores derived using the regression trees models trained on the remaining 4 sets (the SNPs may be divided into more or fewer distinct sets as well). The observed regression coefficient (β_{obs}) of an individual SNP is thus never used directly or indirectly to derive its own gene score weight. Furthermore, as noted above, depending on the nature of the SNP annotations used as predictor variables, accuracy may be improved where the annotations are independent of the tuning dataset. The weights used in gene scores (ŵ_{pred}) are given by the corresponding inverse transformation:

ŵ_{pred}=(βₑₓₜ−ŵ_{pred}*)sign(βₑₓₜ)

Gradient boosted regression trees models were fitted using the “GBM” R package (version 2.1.1) under a Gaussian distribution and squared error loss function. 2,000 trees were fitted with an interaction depth of 5, shrinkage parameter of 0.001 and bag fraction of 0.5. All other parameters were otherwise set to their default values. Fifty-nine independent variables (X₁, X₂, . . . X₅₉) were considered for inclusion in the gradient boosted regression trees models, all of which were derived from sources other than HRS and GENEVA. These comprised measures of association with complex traits such as association p-values, regression coefficients (after standardization for allele frequency) and regional associations. Regional associations were derived from the 100 (herein referred as “regional association”) or 50 (herein referred as “small regional association”) SNPs upstream and downstream of each SNP according to the method previously described (Pare et al., supra). Regional associations reflect the overall impact of a set of contiguous SNPs on genetic variance. The models also included measures of tissue-specific eQTL, which was calculated by summing the −log(p-value) of association of each SNP with genes in each tissue (limiting to significant eQTL associations). Finally, general SNP annotations such as allele frequency in Europeans (from GIANT), LDScore (Bulik-Sullivan et al., supra), regulome score (Boyle, A. P., Hong, E. L., Hariharan, M., Cheng, Y., Schaub, M. A., Kasowski, M., Karczewski, K. J., Park, J., Hitz, B. C., Weng, S., et al. (2012). Annotation of functional variation in personal genomes using RegulomeDB. Genome Res 22, 1790-1797) or linkage disequilibrium with putative disruptive coding mutations (based on CADD score (Kircher, M., Witten, D. M., Jain, P., O'Roak, B. J., Cooper, G. M., and Shendure, J. (2014). A general framework for estimating the relative pathogenicity of human genetic variants. Nature Genetics 46, 310-315)) were included. A full list of independent variables is provided in Table 2 below:

TABLE 2
A list of predictor variables used in boosted regression trees model

| Prediction | | |
|---|---|---|
| variable | URL | Description |
| BMI association | https://www.broadinstitute.org/collaboration/giant/ | -log₁₀(p-value) |
| p-value (GIANT) | index.php/GIANT_consortium_data_files | |
| CAD association | http://www.cardiogramplusc4d.org/ | -log₁₀(p-value) |
| p-value | | |
| (CARDIOGRAM) | | |
| Diabetes | http://diagram-consortium.org/ | -log₁₀(p-value) |
| association p- | | |
| value (DIAGRAM) | | |
| HDL association | http://csg.sph.umich.edu/abecasis/public/lipids2013/ | -log₁₀(p-value) |
| p-value | | |
| Height | https://www.broadinstitute.org/collaboration/giant/ | -log₁₀(p-value) |
| association p- | index.php/GIANT_consortiurn_data_files | |
| value (GIANT) | | |
| LDL association | http://csg.sph.umich.edu/abecasis/public/lipids2013/ | -log₁₀(p-value) |
| p-value | | |
| Total cholesterol | http://csg.sph.umich.edu/abecasis/public/lipids2013/ | -log₁₀(p-value) |
| association p- | | |
| value | | |
| Triglycerides | http://csg.sph.umich.edu/abecasis/public/lipids2013/ | -log₁₀(p-value) |
| association p- | | |
| value | | |
| Diastolic blood | NA | -log₁₀(p-value) |
| pressure | | |
| association p- | | |
| value | | |
| Systolic blood | NA | -log₁₀(p-value) |
| pressure | | |
| association p- | | |
| value | | |
| BMI regression | https://www.broadinstitute.org/collaboration/giant/ | absolute value of |
| coefficient | index.php/GIANT_consortium_data_files | adjusted |
| (GIANT) | | regression |
| | | coefficient |
| CAD regression | http://www.cardiogramplusc4d.org/ | absolute value of |
| coefficient | | adjusted |
| (CARDIOGRAM) | | regression |
| | | coefficient |
| Diabetes | http://diagram-consortium.org/ | absolute value of |
| regression | | adjusted |
| coefficient | | regression |
| (DIAGRAM) | | coefficient |
| HDL regression | http://csg.sph.umich.edu/abecasis/public/lipids2013/ | absolute value of |
| coefficient | | adjusted |
| | | regression |
| | | coefficient |
| Height regression | https://www.broadinstitute.org/collaboration/giant/ | absolute value of |
| coefficient | index.php/GIANT_consortium_data_files | adjusted |
| (GIANT) | | regression |
| | | coefficient |
| LDL regression | http://csg.sph.umich.edu/abecasis/public/lipids2013/ | absolute value of |
| coefficient | | adjusted |
| | | regression |
| | | coefficient |
| Total cholesterol | http://csg.sph.umich.edu/abecasis/public/lipids2013/ | absolute value of |
| regression | | adjusted |
| coefficient | | regression |
| | | coefficient |
| Triglycerides | http://csg.sph.umich.edu/abecasis/public/lipids2013/ | absolute value of |
| regression | | adjusted |
| coefficient | | regression |
| | | coefficient |
| Diastolic blood | NA | absolute value of |
| pressure | | adjusted |
| regression | | regression |
| coefficient | | coefficient |
| Systolic blood | NA | absolute value of |
| pressure | | adjusted |
| regression | | regression |
| coefficient | | coefficient |
| Regulome score | http://www.regulomedb.org/ | * (see below) |
| Adipose | http://www.gtexportal.org/home/ | sum of -log(p- |
| Subcutaneous | | value) of eQTL |
| eQTL p-value | | associations |
| Artery Aorta | http://www.gtexportal.org/home/ | sum of -log(p- |
| eQTL p-value | | value) of eQTL |
| | | associations |
| Artery Tibial | http://www.gtexportal.org/home/ | sum of -log(p- |
| eQTL p-value | | value) of eQTL |
| | | associations |
| Esophagus | http://www.gtexportal.org/home/ | sum of -log(p- |
| Mucosa eQTL p- | | value) of eQTL |
| value | | associations |
| Esophagus | http://www.gtexportal.org/home/ | sum of -log(p- |
| Muscularis eQTL | | value) of eQTL |
| p-value | | associations |
| Heart Left | http://www.gtexportal.org/home/ | sum of -log(p- |
| Ventricle eQTL p- | | value) of eQTL |
| value | | associations |
| Lung eQTL p- | http://www.gtexportal.org/home/ | sum of -log(p- |
| value | | value) of eQTL |
| | | associations |
| Muscle Skeletal | http://www.gtexportal.org/home/ | sum of -log(p- |
| eQTL p-value | | value) of eQTL |
| | | associations |
| Nerve Tibial | http://www.gtexportal.org/home/ | sum of -log(p- |
| eQTL p-value | | value) of eQTL |
| | | associations |
| Skin Sun Exposed | http://www.gtexportal.org/home/ | sum of -log(p- |
| Lower leg eQTL | | value) of eQTL |
| p-value | | associations |
| Stomach eQTL p- | http://www.gtexportal.org/home/ | sum of -log(p- |
| value | | value) of eQTL |
| | | associations |
| Thyroid eQTL p- | http://www.gtexportal.org/home/ | sum of -log(p- |
| value | | value) of eQTL |
| | | associations |
| Whole Blood | http://www.gtexportal.org/home/ | sum of -log(p- |
| eQTL p-value | | value) of eQTL |
| | | associations |
| BMI Minor Allele | https://www.broadinstitute.org/collaboration/giant/ | Minor allele |
| Frequency | index.php/GIANT_consortium_data_files | frequency |
| (GIANT) | | |
| HDL Minor Allele | http://csg.sph.umich.edu/abecasis/public/lipids2013/ | Minor allele |
| Frequency | | frequency |
| Height Minor | https://www.broadinstitute.org/collaboration/giant/ | Minor allele |
| Allele Frequency | index.php/GIANT_consortium_data_files | frequency |
| (GIANT) | | |
| LDScore | https://github.com/bulik/Idsc/wiki/LD-Score- | ** (see below) |
| | Estimation-Tutorial | |
| BMI regional | https://www.broadinstitute.org/collaboration/giant/ | See paragraph |
| genetic variance | index.php/GIANT_consortium_data_files | above |
| Height regional | https://www.broadinstitute.org/collaboration/giant/ | See paragraph |
| genetic variance | index.php/GIANT_consortium_data_files | above |
| Diastolic blood | NA | See paragraph |
| pressure regional | | above |
| genetic variance | | |
| Diabetes regional | http://diagram-consortium.org/ | See paragraph |
| genetic variance | | above |
| (DIAGRAM) | | |
| HDL regional | http://csg.sph.umich.edu/abecasis/public/lipids2013/ | See paragraph |
| genetic variance | | above |
| Systolic blood | NA | See paragraph |
| pressure regional | | above |
| genetic variance | | |
| Total cholesterol | http://csg.sph.umich.edu/abecasis/public/lipids2013/ | See paragraph |
| regional genetic | | above |
| variance | | |
| Triglycerides | http://csg.sph.umich.edu/abecasis/public/lipids2013/ | See paragraph |
| regional genetic | | above |
| variance | | |
| LDL regional | http://csg.sph.umich.edu/abecasis/public/lipids2013/ | See paragraph |
| genetic variance | | above |
| CAD regional | http://www.cardiogramplusc4d.org/ | See paragraph |
| genetic variance | | above |
| (CARDIOGRAM) | | |
| BMI small | https://www.broadinstitute.org/collaboration/giant/ | See paragraph |
| regional genetic | index.php/GIANT_consortium_data_files | above |
| variance | | |
| Height small | https://www.broadinstitute.org/collaboration/giant/ | See paragraph |
| regional genetic | index.php/GIANT_consortium_data_files | above |
| variance | | |
| Diastolic blood | NA | See paragraph |
| pressure small | | above |
| regional genetic | | |
| variance | | |
| Diabetes small | http://diagram-consortium.org/ | See paragraph |
| regional genetic | | above |
| variance | | |
| (DIAGRAM) | | |
| HDL small | http://csg.sph.umich.edu/abecasis/public/lipids2013/ | See paragraph |
| regional genetic | | above |
| variance | | |
| Systolic blood | NA | See paragraph |
| pressure small | | above |
| regional genetic | | |
| variance | | |
| Total cholesterol | http://csg.sph.umich.edu/abecasis/public/lipids2013/ | See paragraph |
| small regional | | above |
| genetic variance | | |
| Triglycerides | http://csg.sph.umich.edu/abecasis/public/lipids2013/ | See paragraph |
| small regional | | above |
| genetic variance | | |
| LDL small | http://csg.sph.umich.edu/abecasis/public/lipids2013/ | See paragraph |
| regional genetic | | above |
| variance | | |
| CAD small | http://www.cardiogramplusc4d.org/ | See paragraph |
| regional genetic | | above |
| variance | | |
| (CARDIOGRAM) | | |
| CADD score | http://cadd.gs.washington.edu/download | *** |

*: The original Regulome score values of “7, 6, 5, 4, 3b, a3, 2c, 2b, 2a, 1f, 1e, 1d, 1c, 1b, 1a and lnf” are replaced by 0, 1, 2, 3, 4, 5, 6, 7, 8, 9, 10, 11, 12, 13, 14, 15, respectively
**: For any SNP, if its LDscore is not available, its score is set to 1
***: Calculated according to the following procedure: (1) The CADD score of each SNP is downloaded; (2) for any SNP, if it is not located in an exonic area, the CADD score is set to 0; (3) The final score is determined for each SNP summing the product of the CADD score and LD r² of SNPs within 100 Kb of that SNP i.e. score = sum(r² * CADD score).

To avoid including too many non-informative variables in the regression trees, each predictor variable was first tested for association with w_{pred}* in standard linear regression models and only those with marginal association p-value<0.05 were retained for gradient boosted regression trees modeling.

A few limitations are noted. First, the methods 200 and 200A described above are based on the premise that SNPs contribute additively to genetic variance. While empirical evidence suggests this holds true in most cases, the effectiveness of the methods 200 and 200A may be hampered in genomic regions where strong genetic interactions are present (e.g. HLA), and alternative methods such as LDpred might be better suited (Vilhjalmsson et al., supra). Second, there is a possibility that gene scores derived using the methods 200 and 200A are inherently population-specific. However, with the exception of unadjusted gene scores, all methods require the determination of parameters in the prediction target population and the methods 200 and 200A are no different. Furthermore, if the genetic architecture varies between populations, then no gene score will perform universally well and it will be beneficial to tailor gene scores to each population. The observation that the boosted regression tree implementation of the method 200A outperformed other methods in GENEVA suggests at least a part of the improvement is transferable to other populations. Third, the correction for LD yielded advantageous results yet is expected to lead to some loss of information when SNPs are in partial LD. This loss of information was estimated at ˜12% of genetic variance in simulations using real-life haplotypes from the 1000G Project. Nonetheless, the above-described correction method for LD also has several benefits such as simplicity, use of summary association statistics and intrinsic robustness to minor misspecification of LD or association strength. The above-described correction method for LD is merely one exemplary method for correcting the genotypic weightings in a predictive gene score for LD; other approaches to correcting for LD, whether now known or hereafter developed, may also be used.

In summary, the exemplary methods 200 and 200A described above may be applied to improve the prediction of polygenic traits using gene scores. The test results show that for the classic polygenic traits height and BMI, 46.6% and 44.4% of the estimated polygenic genetic variance can be captured by gene scores generated using boosted regression tree implementations of these methods. These results demonstrate the potential of such machine-learning methods to harness the considerable amount of information from large genetic meta-analyses and the usefulness of integrating annotation data into gene scores. Indeed, the improvement observed for height and BMI is all the more remarkable as both HRS and GENEVA have relatively small sample sizes by standards of genetic epidemiological studies. As with other prediction problems involving machine-learning techniques, incremental improvements are to be expected with increases in sample size, use of more informative predictor variables and availability of better summary association statistics.

As can be seen from the above description, the methods for developing predictive gene scores as described herein represent significantly more than merely using categories to organize, store and transmit information and organizing information through mathematical correlations. The methods for developing predictive gene scores are in fact an improvement to the technology of eukaryote genomic prediction, as they define logical structures and processes that provide an improvement in computer capabilities in tuning predictive associations to a target population when developing predictive gene scores. Moreover, because the methods described herein require linear partitioning of sequentially arranged data, these methods are limited to operations on the eukaryote genome, which possesses the required linear, sequential arrangement of data. As such, the present technology is confined to eukaryote genomic prediction applications, and more particularly to computerized implementations of eukaryote genomic prediction in machine learning applications.

The present technology may be embodied within a system, a method, a computer program product or any combination thereof. The computer program product may include a computer readable storage medium or media having computer readable program instructions thereon for causing a processor to carry out aspects of the present technology. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present technology may be assembler instructions, instruction-set-architecture (ISA) instructions, machine instructions, machine dependent instructions, microcode, firmware instructions, state-setting data, or either source code or object code written in any combination of one or more programming languages, including an object oriented programming language or a conventional procedural programming language. The computer readable program instructions may execute entirely on the user's computer, partly on the user's computer, as a stand-alone software package, partly on the user's computer and partly on one or more remote computers or entirely on the remote computer(s) or server(s). In the latter scenario, the remote computer(s) may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to implement aspects of the present technology.

Aspects of the present technology have been described above with reference to flowchart illustrations and/or block diagrams of methods, apparatus (systems) and computer program products according to various embodiments. In this regard, the flowchart and block diagrams in the Figures illustrate the architecture, functionality, and operation of possible implementations of systems, methods and computer program products according to various embodiments of the present technology. For instance, each block in the flowchart or block diagrams may represent a module, segment, or portion of instructions, which comprises one or more executable instructions for implementing the specified logical function(s). It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Some specific examples of the foregoing have been noted above but any such noted examples are not necessarily the only such examples. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It also will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the to flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

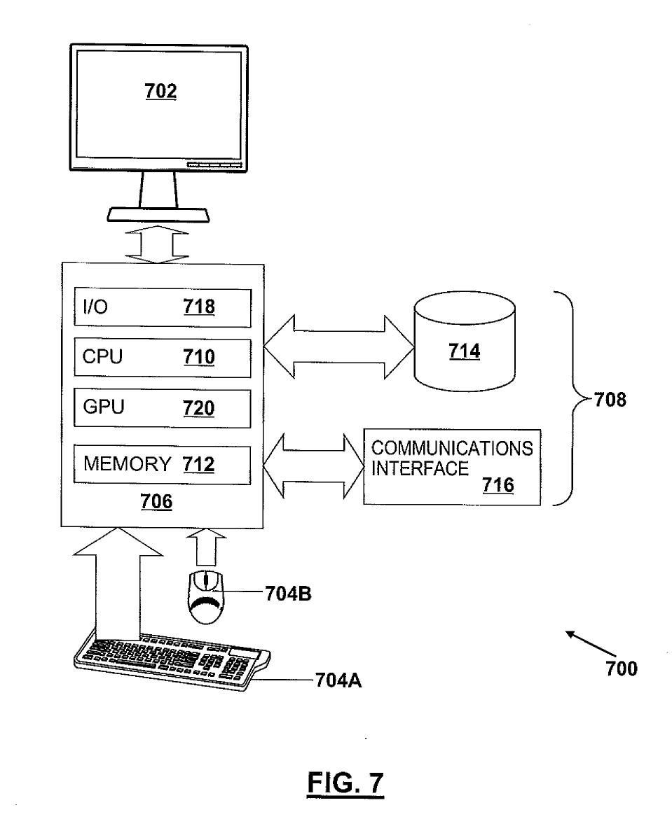

An illustrative computer system in respect of which the technology herein described may be implemented is presented as a block diagram in FIG. 7. The illustrative computer system is denoted generally by reference numeral 700 and includes a display 702, input devices in the form of keyboard 704A and pointing device 704B, computer 706 and external devices 708. While pointing device 704B is depicted as a mouse, it will be appreciated that other types of pointing device, including for example a touch-screen interface, may also be used.

The computer 706 may contain one or more processors or microprocessors, such as a central processing unit (CPU) 710. The CPU 710 performs arithmetic calculations and control functions to execute software stored in an internal memory 712, preferably random access memory (RAM) and/or read only memory (ROM), and possibly additional memory 714. The additional memory 714 may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This additional memory 714 may be physically internal to the computer 706, or external as shown in FIG. 7, or both.

The computer system 700 may also include other similar means for allowing computer programs or other instructions to be loaded. Such means can include, for example, a communications interface 716 which allows software and data to be transferred between the computer system 700 and external systems and networks. Examples of communications interface 716 can include a modem, a network interface such as an Ethernet card, a wireless communication interface, or a serial or parallel communications port. Software and data transferred via communications interface 716 are in the form of signals which can be electronic, acoustic, electromagnetic, optical or other signals capable of being received by communications interface 716. Multiple interfaces, of course, can be provided on a single computer system 700.

Input and output to and from the computer 706 is administered by the input/output (I/O) interface 718. This I/O interface 718 administers control of the display 702, keyboard 704A, external devices 708 and other such components of the computer system 700. The computer 706 also includes a graphical processing unit (GPU) 720. The latter may also be used for computational purposes as an adjunct to, or instead of, the (CPU) 710, for mathematical calculations.

The various components of the computer system 700 are coupled to one another either directly or by coupling to suitable buses.

The term “computer system” and related terms, as used herein, is not limited to any particular type of computer system and encompasses servers, desktop computers, laptop computers, networked mobile wireless telecommunication computing devices such as smartphones, tablet computers, as well as other types of computer systems.

Thus, computer readable program code for implementing aspects of the technology described herein may be contained or stored in the memory 712 of the computer 706, or on a computer usable or computer readable medium external to the computer 706, or on any combination thereof.

Finally, the terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description has been presented for purposes of illustration and description, but is not intended to be exhaustive or limited to the form disclosed. Many modifications and variations will be apparent to those of ordinary skill in the art without departing from the scope of the claims. The embodiment was chosen and described in order to best explain the principles of the technology and the practical application, and to enable others of ordinary skill in the art to understand the technology for various embodiments with various modifications as are suited to the particular use contemplated.

Certain currently preferred embodiments have been described by way of example. It will be apparent to persons skilled in the art that a number of variations and modifications can be made without departing from the scope of the claims. In construing the claims, it is to be understood that the use of a computer to implement the embodiments described herein is essential.

What is claimed is:

1. A computer-implemented method for developing a predictive gene score for a target biological characteristic in a prediction target population, the method comprising:
receiving a baseline dataset;
the baseline dataset comprising a first plurality of associations between:
genetic variants of sequence elements of a subject genome; and
the target biological characteristic;
receiving a tuning dataset;
the tuning dataset comprising, for a representative sample of the prediction target population, a second plurality of associations between:
genetic variants of the sequence elements of the subject genome; and
the target biological characteristic;
wherein the associations in the baseline dataset and the tuning data set are genotypic weightings representing contributions of the respective genetic variants to a value of the target biological characteristic;
notionally partitioning the subject genome into s discrete contiguous genome segments where s≥2 so that, for each genome segment, the subject genome notionally comprises:
that genome segment; and
the remainder of the subject genome other than and excluding that genome segment;
obtaining adjusted associations for each genome segment without overfitting by, for each genome segment:
using only the associations for the sequence elements in the remainder of the subject genome to derive a tuning function for that genome segment;
wherein the tuning function maps the associations in the baseline dataset for the remainder of the subject genome to respective corresponding ones of the associations in the tuning dataset for the remainder of the subject genome;
applying the tuning function to the associations in the baseline dataset to obtain adjusted associations for the sequence elements in that genome segment;
wherein the adjusted associations are tuned to the prediction target population; and
using the adjusted associations to form the predictive gene score for the target biological characteristic in the prediction target population.

2. The method of claim 1, wherein using only the associations for the sequence elements in the remainder of the subject genome to derive a tuning function for that genome segment comprises deriving regression trees representing the tuning function.

3. The method of claim 1, further comprising:
receiving at least one annotation, wherein each annotation is associated with a respective sequence element; and
for each genome segment for which the remainder of the subject genome includes a sequence element with which one of the at least one annotation is associated, using each such annotation in deriving the tuning function for that genome segment.

4. The method of claim 3, wherein the at least one annotation is selected from the group consisting of BMI association p-value, CAD association p-value, diabetes association p-value, HDL association p-value, height association p-value, LDL association p-value, total cholesterol association p-value, triglycerides association p-value, diastolic blood pressure association p-value, systolic blood pressure association p-value, BMI regression coefficient, CAD regression coefficient, diabetes regression coefficient, HDL regression coefficient, height regression coefficient, LDL regression coefficient, total cholesterol regression coefficient, triglycerides regression coefficient, diastolic blood pressure regression coefficient, systolic blood pressure regression coefficient, regulome score, adipose subcutaneous eQTL p-value, artery aorta eQTL p-value, artery tibial eQTL p-value, esophagus mucosa eQTL p-value, esophagus muscularis eQTL p-value, heart left ventricle eQTL p-value, lung eQTL p-value, muscle skeletal eQTL p-value, nerve tibial eQTL p-value, skin sun exposed lower leg eQTL p-value, stomach eQTL p-value, thyroid eQTL p-value, whole Blood eQTL p-value, BMI minor allele frequency, HDL minor allele frequency, height minor allele frequency, LDScore, BMI regional genetic variance, height regional genetic variance, diastolic blood pressure regional genetic variance, diabetes regional genetic variance, IIDL regional genetic variance, systolic blood pressure regional genetic variance, total cholesterol regional genetic variance, triglycerides regional genetic variance, LDL regional genetic variance, CAD regional genetic variance, BMI small regional genetic variance, height small regional genetic variance, diastolic blood pressure small regional genetic variance, diabetes small regional genetic variance, HDL small regional genetic variance, systolic blood pressure small regional genetic variance, total cholesterol small regional genetic variance, triglycerides small regional genetic variance, LDL small regional genetic variance, CAD small regional genetic variance and CADD score.

5. The method of claim 3, wherein using only the associations for the sequence elements in the remainder of the subject genome to derive a tuning function for that genome segment comprises deriving regression trees representing the tuning function.

6. A computer-implemented method for tuning associations between genetic variants of sequence elements of a subject genome and a target biological characteristic to a target population, the method comprising:
notionally partitioning the subject genome into discrete contiguous genome segments; and
deriving a tuning function for each genome segment, the tuning function tuning the associations to the target population;
wherein derivation of the tuning function for each genome segment excludes sequence elements in that same genome segment to avoid overfitting.
